(19) United States
(12) Patent Application Publication
(10) Pub. No.: US 2026/0021024 A1
(43) Pub. Date: Jan. 22, 2026

(54) PRODUCTS AND METHODS FOR THE TREATMENT OF A SKIN CONDITION

(71) Applicant: Moran SHARONI, Kadima (IL)

(72) Inventor: Moran SHARONI, Kadima (IL)

(21) Appl. No.: 18/863,662

(22) PCT Filed: Mar. 27, 2024
(86) PCT No.: PCT/IL2024/050316

§ 371 Date: Nov. 7, 2024

(30) Foreign Application Priority Data
Mar. 27, 2023 (IL) 301726

(51) Int. Cl. A61K 8/02; A61Q 19/02

(52) U.S. Cl. CPC A61K 8/022; A61Q 19/02; A61K 2800/522; A61K 2800/5922; A61K 2800/87

(57) ABSTRACT

Provided is a skin-care product comprising a sealed, liquid-permeable container containing a composition in the form of a powder, the powder comprising a dermatologically effective material, wherein the powder is configured to form a paste upon contact with water, and wherein the container is configured to release at least a portion of the formed paste.

### FIELD OF THE INVENTION

The invention, in some embodiments, relates to the field of topical treatment of skin conditions, and more particularly to skin-care products comprising a powder contained within a sealed, liquid-permeable container and methods of use thereof.

### BACKGROUND

The skin, which is the largest organ of the body, serves to cover and protect the body. Skin conditions may clog, irritate or inflame the skin, and may cause rashes or other changes in the appearance of the skin.

Common skin conditions include psoriasis, acne, dermatitis, rosacea, eczema and wrinkles.

### SUMMARY OF THE INVENTION

Some embodiments of the invention relate to skin-care products comprising a sealed, liquid-permeable container containing a composition in the form of a powder, the powder comprising a dermatologically effective material, wherein the powder is configured to form a paste upon contact with water, and wherein the container is configured to release at least a portion of the formed paste.

The present invention provides an easy to use product for topical use in treatment of a skin condition, which is provided in a pre-measured amount, which is stable, easily portable and easy to store.

Aspects and embodiments of the invention are described in the specification herein below and in the appended claims.

According to an aspect of some embodiments of the present invention, there is provided a skin-care product comprising a sealed, liquid-permeable container containing a composition in the form of a powder, the powder comprising a dermatologically effective material, wherein the powder is configured to form a paste upon contact with water, and wherein the container is configured to release at least a portion of the formed paste.

According to some embodiments, water for contacting with the powder may be provided at any temperature, such as at room temperature, at a temperature less than room temperature or at a temperature above room temperature.

According to some embodiments, the dermatologically effective material is selected from the group consisting of a dermatologically compatible clay, a dried plant material and/or extract thereof, an antioxidant, a and a keratolytic agent and combinations thereof.

According to some embodiments, the clay is selected from the group consisting of kaolin clay, bentonite clay, French green clay, French rose clay, Fuller's earth clay, Rhassoul clay and combinations thereof. According to some such embodiments, the clay comprises bentonite clay and kaolin clay.

According to some embodiments, the dried plant material and/or extract thereof is selected from the group consisting of rooibos, chamomile, spearmint, nettle, oregano, rosemary, dandelion, sage, arrowroot, ginger, gingko, ginseng, hibiscus, basil, parsley, Calendula officinalis, aloe vera, Echinacea, Comfrey, lavender, violet, thyme, cinnamon, helichrysum, Camellia sinensis and combinations thereof. According to some such embodiments, the dried plant material and/or extract thereof comprises dried leaves and extract of a rooibos plant, chamomile extract, green tea extract and Calendula officinalis extract.

According to some embodiments, the antioxidant is selected from the group consisting of vitamin C, niacinamide, resveratrol, vitamin E, retinol, butylated hydroxyanisole (BHA), a carotene, ubiquinol (coenzyme Q), lipoic acid, uric acid, gluthianone, a polyphenol, pomegranate extract and combinations thereof.

According to some embodiments, the keratolytic agent is selected from the group consisting of salicylic acid, urea, citric acid, polyhydroxy acid, lactic acid, allantoin, glycolic acid, trichloroacetic acid and combinations thereof.

According to some embodiments, the powder composition comprises pomegranate extract and salicylic acid.

According to some embodiments, the powder composition comprises kaolin clay, bentonite clay, Aspalathus Linearis (Rooibos) Leaves, Aspalathus Linearis (Rooibos) Leaf Extract, Chamomilla Recutita (Chamomile) Flower Extract, Salicylic Acid, Maltodextrin, Punica Granatum Pericarp (Pomegranate) Extract, Calendula officinalis Flower Extract, Camellia sinensis (Green Tea) Leaf Extract.

According to some embodiments, the liquid-permeable container comprises a porous, sealed bag. According to some such embodiments, the bag comprises expandable pores, wherein upon expansion of the pores, the paste exits the bag through the pores.

According to some embodiments, the pores are expandable upon contact of said bag with water. According to some embodiments, the pores are expandable upon exertion of manual pressure upon said bag by a user. According to some embodiments, the bag is elastic, thereby expanding said pores upon applying traction power thereon.

According to some embodiments, the paste formed upon contact with water occupies a greater amount of the inner volume of the bag than is occupied by the powder prior to contact with water. According to some embodiments, the powder is provided in an amount which sufficiently fills an inner volume of the bag, such that upon formation of the paste, which occupies a greater volume that the powder, the inner volume of the bag is substantially filled, wherein outward pressure is exerted upon the bag causing expansion of the pores. According to some such embodiments, the powder is provided in an amount which substantially fills an entire inner volume of the bag.

According to an aspect of some embodiments of the present invention, there is provided the skin-care product as disclosed herein, for use in topical treatment of a skin condition.

According to an aspect of some embodiments of the present invention, there is provided a method of treating a skin condition in a subject in need thereof, comprising providing the skin-care product as disclosed herein, contacting the skin-care product with water wherein a paste is formed, and applying the paste to at least a portion of the skin of the subject.

According to some embodiments the skin condition is selected from the group consisting of acne, eczema, psoriasis, dermatitis, rosace, an age-related skin condition, and combinations thereof.

As used herein, the term “skin condition” relates to any medical or cosmetic condition that affects the skin or a portion of the skin of a subject.

As used herein, the term “treating” includes ameliorating, mitigating, and reducing the instances of a disease or condition, or the symptoms of a disease or condition. In some embodiments, the treating refers to medical treatment i.e. treatment relating to the health of the subject. In some embodiments, the treating refers to cosmetic treatment i.e. treatment relating to aesthetic, non-medical aspects of a skin condition.

As used herein, the term “dermatologically effective amount” means the amount of an active substance that, when topically applied to at least a portion of the skin of a subject for treating a disease, disorder, or other undesirable cosmetic or medical condition, is sufficient to have a beneficial effect with respect to that disease, disorder, or condition. The dermatologically effective amount will vary depending on the chemical identity and formulation form of the active substance, the disease or condition and its severity, and the age, and other relevant characteristics of the patient to be treated. Determining the therapeutically effective amount of a given active substance is within the ordinary skill of the art and typically requires no more than routine experimentation.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10% of that value.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A and 1B schematically illustrate a skin-care product in accordance with the principles of the present invention, comprising a sealed bag which is opaque (as shown in FIG. 1A) or transparent (as shown in FIG. 1B) containing a powder composition.

FIGS. 2A and 2B schematically illustrate the skin-care products of FIGS. 1A, 1B, respectively, comprising a paste formed upon contact of the powder composition with water.

FIGS. 3A and 3B schematically illustrate the impact of immersion in water on the bag of the skin-care products of FIGS. 1A and 1B.

FIGS. 4A and 4B schematically illustrate the skin-care products of FIGS. 1A and 1B, further comprising a string and tab.

### DESCRIPTION OF SOME EMBODIMENTS OF THE INVENTION

Some embodiments of the invention relate to skin-care products and uses thereof in the treatment of skin conditions.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

According to an aspect of some embodiments of the present invention, there is provided a skin-care product comprising a sealed, liquid-permeable container containing a composition in the form of a powder, the powder comprising a dermatologically effective material, wherein the powder is configured to form a paste upon contact with water, and wherein the container is configured to release at least a portion of the formed paste.

According to some embodiments, the dermatologically effective material is selected from the group consisting of a dermatologically compatible clay, a dried plant material and/or extract thereof, an antioxidant, a keratolytic agent.

According to some embodiments, the dermatologically compatible clay is selected from the group consisting of kaolin clay, bentonite clay, French green clay, French rose clay, Fuller's earth clay, Rhassoul clay and combinations thereof. According to a preferred embodiment, the clay comprises kaolin clay. According to a preferred embodiment, the clay comprises bentonite clay and kaolin clay. According to some embodiments, the clay provides a detoxifying effect.

According to some embodiments, the dermatologically effective material is a dried plant material and/or an extract thereof. According to some such embodiments, the dried plant material and/or extract thereof is from a plant selected from the group consisting of rooibos, chamomile, spearmint, nettle, oregano, rosemary, dandelion, sage, arrowroot, ginger, gingko, ginseng, hibiscus, basil, parsley, Calendula officinalis extract, aloe vera, Echinacea, Comfrey, lavender, violet, thyme, cinnamon, helichrysum, Camellia sinensis and combinations thereof. According to a preferred embodiment, the dried plant material comprises dried leaves and extract of a rooibos plant, chamomile extract, green tea extract and Calendula officinalis extract. According to some embodiments, the rooibos plant leaves and extract, chamomile extract, and green tea extract reduce redness of the skin, while the Calendula officinalis extract promotes radiance (i.e. provides a glowing appearance to the skin). According to some embodiments, the antioxidant is selected from the group consisting of vitamin C, niacinamide, resveratrol, vitamin E, retinol, butylated hydroxyanisole (BHA), a carotene, ubiquinol (coenzyme Q), lipoic acid, uric acid, gluthianone, a polyphenol, pomegranate extract and combinations thereof. According to a preferred embodiment, the antioxidant comprises pomegranate extract. According to a preferred embodiment, the pomegranate extract serves to restore the homeostasis of acneic skin, to reduce blemishes.

According to some such embodiments, the keratolytic agent is selected from the group consisting of salicylic acid, urea, citric acid, polyhydroxy acid, lactic acid, allantoin, glycolic acid, trichloroacetic acid and combinations thereof. According to a preferred embodiment, the keratolytic agent comprises salicylic acid.

According to some embodiments, the antioxidant and/or the keratolytic agent reduce blemishes.

According to some embodiments, the composition further comprise a carrier, such as maltodextrin.

According to some embodiments, the composition further comprises a vitamin, an antioxidant, a hydroxy acid, hyaluronic acid, dimethylaminoethanol (DMAE) and combinations thereof.

According to some embodiments, the vitamin is selected from the group consisting of vitamin A (retinoid, such as retinol and/or retinoic acid); vitamin B; vitamin C (ascorbic acid); vitamin D; vitamin E; vitamin F; vitamin K; and combinations thereof. According to some such embodiments, the vitamin B is selected from the group consisting of B1 (thiamine), B2 (riboflavin), B3 (niacin, such as niacinamide and/or nicotinic acid), B5 (pantothenic acid), B6, B7 (biotin), B9 (folate), B12 and combinations thereof. According to a preferred embodiment, the vitamin comprises vitamin B3 in the form of niacinamide.

According to some such embodiments, the vitamin B is selected from the group consisting of B1 (thiamine), B2 (riboflavin), B3 (niacin), B5 (pantothenic acid), B6, B7 (biotin), B9 (folate), B12 and combinations thereof.

According to some embodiments, the composition further comprises a preservative. According to some embodiments, the preservative is selected from the group consisting of potassium sorbate, sodium benzoate methyl hydroxybenzoate, propyl hydroxybenzoate, chlorocresol, benzoic acid, phenyl mercuric nitrate, benzalkonium chloride, chlorhexidine acetate, benzyl alcohol, hoelen and combinations thereof. According to a preferred embodiment, the preservative is selected from the group consisting of potassium sorbate, sodium benzoate, hoelen and combinations thereof.

According to some embodiments, the composition further comprises a skin-softening agent. According to some such embodiments, the skin-softening agent comprises succinic acid.

According to some embodiments, the composition further comprises a starch. According to some such embodiments, the starch is selected from the group consisting of tapioca starch, corn starch, rice starch and combinations thereof. According to a preferred embodiment, the starch comprises tapioca starch.

According to some embodiments, the powder composition comprises kaolin clay, bentonite clay, Aspalathus Linearis (Rooibos) Leaves, Aspalathus Linearis (Rooibos) Leaf Extract, Chamomilla Recutita (Chamomile) Flower Extract, Salicylic Acid, Maltodextrin, Punica Granatum Pericarp (Pomegranate) Extract, Calendula officinalis Flower Extract, Camellia sinensis (Green Tea) Leaf Extract.

According to some embodiments, the composition further comprises an excipient, such as, for example, a thickener, a gelling agent, a neutralizer, an occlusive, an antioxidant, a buffering agent, a pH adjusting agent, a filler, an emulsifier, a co-emulsifier, an emollient, a solvent, a stabilizing agent, a solubilizer, a stiffening agent, a suspending agent, a binder, a viscosity-increasing agent, a penetration enhancer, a preservative, a chelating agent, a disintegrant, a plasticizer, a humectant, a fragrance and the like.

According to some embodiments, the composition is substantially devoid of aluminum (Alcloxa), oxybenzone, alcohol, witch hazel, tocopheryl acetate, menthol, petroleum, sodium laureth sulfate, sodium lauryl sulfate, palm oil, sodium chloride, Formaldehyde, sodium C14-16 olefin sulfate, octocrylene, 4MBC, butylparaben, fragrance, essential oils and mineral oils.

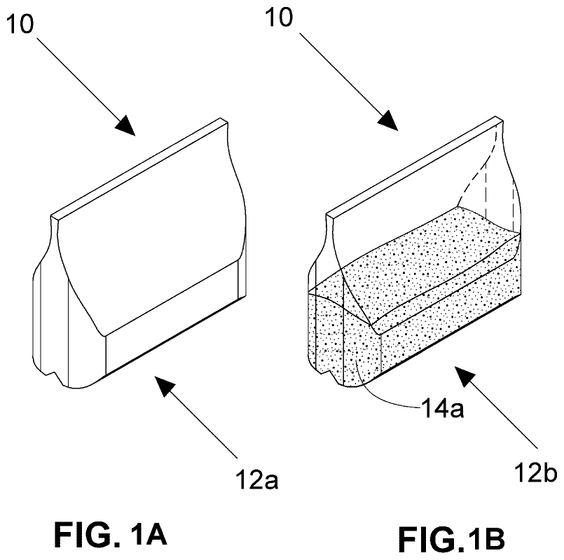

Referring now to FIGS. 1A, 1B, there is schematically shown a skin-care product 10 in accordance with the principles of the present invention. Skin-care product 10 comprises a sealed, liquid-permeable container 12a, 12b, in the form of a porous, sealed bag or pouch. Container 12a, 12b contains a composition in the form of a powder 14a (shown in FIG. 1B), powder 14a comprising a dermatologically effective material. According to an embodiment, as shown in FIG. 1A, container 12a is opaque. According to an alternative embodiment, as shown in FIG. 1B, container 12b is transparent. According to an alternative embodiment (not shown), the bag may be partially transparent, such as comprising a transparent portion or window through with the contents of the bag may be viewed.

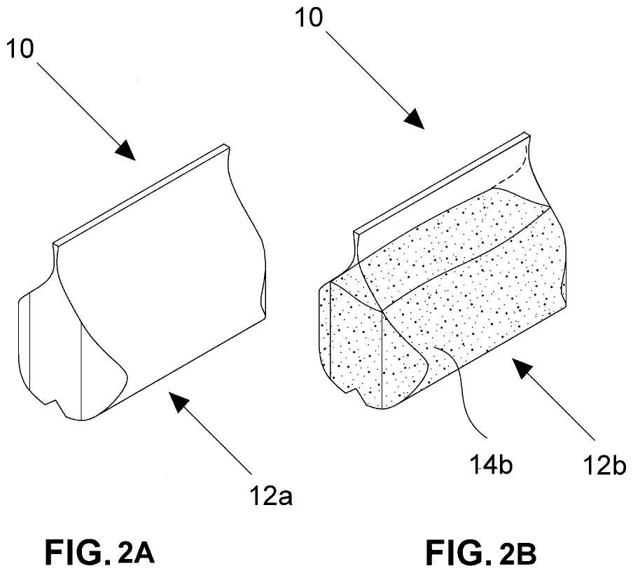

As shown in FIGS. 2A, 2B, upon contact with water, powder 14a forms a paste 14b (seen in FIG. 2B), wherein a volume of paste 14b is greater than that of powder 14a, and wherein container 12a, 12b releases at least a portion of formed paste 14b.

The bag may be formed from any suitable, dermatologically-compatible material, such as a material from which a teabag is generally made, such as silk, nylon, filter paper, corn fiber or food-grade plastics. Optionally, the material from which the bag is formed biodegradable and/or compostable. The bag may be provided in any suitable shape, such as a square, rectangular, circular, triangular, heart, etc. shape. The bag may be provided in any size, such as having dimensions configured to contain a sufficient amount of the powder composition to provide, when contacted with water, an amount of paste which is sufficient for application to a desired area of skin, such as an area of the face and/or body of a subject. According to some embodiments, the amount of powder provided within a bag is in the range of from about 2 to 25 g, such as about 2 g, about 3 g, about 4 g, 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g or about 25 g. According to some embodiments, the amount is in the range of from about 5 to 15 g. According to some embodiments, the powder is contacted with about 3 tablespoons water (a ratio of about 5:15 g/ml powder:water) in order to provide the paste, for use as a face mask or as a targeted spot treatment. According to some embodiments, the powder is contacted with any suitable amount of water to provide a desired consistency for use as a body treatment, such as addition of about 15 g powder to sufficient water in a bathtub into which the body of the subject can be immersed.

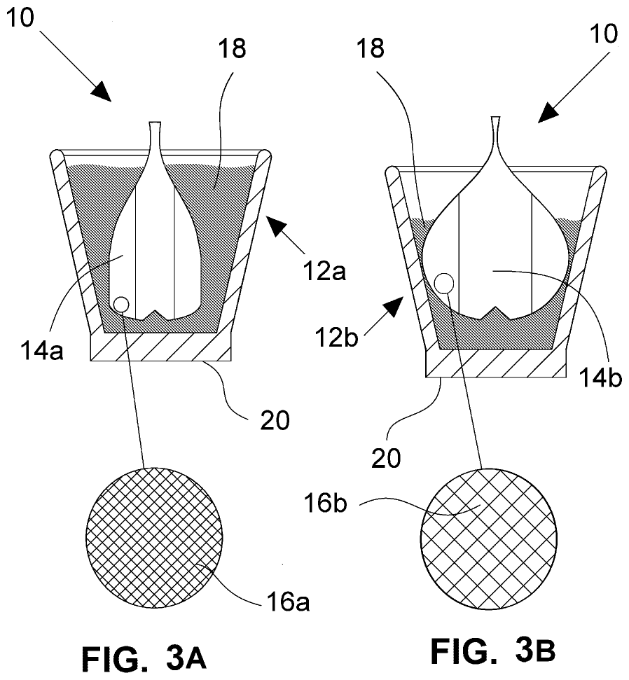

According to an embodiment, as shown in FIGS. 3A, 3B, container 12b comprises pores 16a having an initial circumference size. During use, container 12b is contacted with water 18, such as by immersion in water 18 provided within a vessel 20, whereupon pores 16a of expand to form expanded pores 16b, having a greater circumference size than that of pores 16a, wherein paste 14b can exit container 12b through expanded pores 16b.

According to some such embodiments, the container is formed from a material wherein the pores are expandable upon contact of the bag with water.

According to some embodiments, the pores are expandable due to pressure exerted on the inner surface of the walls of the container by the paste following expansion upon contact with water.

According to some such embodiments, the pores are expandable upon exertion of manual pressure upon the bag by a user, such as by pressing or squeezing the bag.

According to some embodiments, the bag is formed from an elastic material, wherein said pores expand upon applying traction power thereon.

According to some embodiments, the powder composition is provided in an amount which substantially fills an entire inner volume of said bag, and the formed paste occupies a greater volume than is occupied by the powder, such that upon formation of the paste, outward pressure is exerted upon the bag by the paste, causing expansion of the pores.

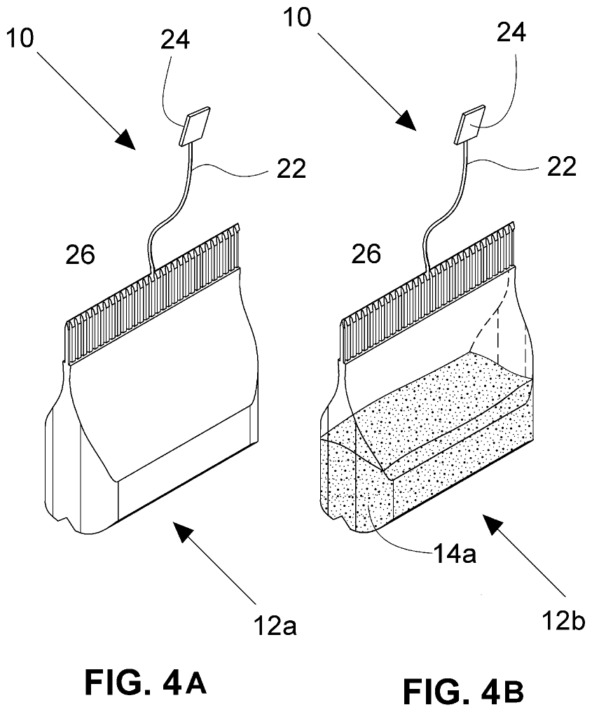

As shown in FIGS. 4A, 4B, container 12A, 12B optionally further comprises an elongated extension 22, such as a string, thread or cord attached to an upper portion or edge 26, which may be gripped by a user for ease of removal of container 12A, 12B from vessel 22 after use. Elongated extension may optionally further comprise a tab 24 at an end distal from the point of attachment to upper portion or edge 26.

According to an aspect of some embodiments of the present invention, there is provided the skin-care product as disclosed herein for topical use in treatment of a skin condition.

According to an aspect of some embodiments of the present invention, there is provided a method of treating a skin condition in a subject in need thereof, comprising providing the skin-care product as disclosed herein, contacting the skin-care product with water wherein a paste is formed, and applying the paste to at least a portion of the skin of the subject.

According to some embodiments, the paste is applied in the form of a mask, such as a face mask or a body mask. The paste may be applied by spreading over an area of skin to be treated, either by squeezing directly from the container, or by using the fingers or any suitable applicator, such as brush, spatula or sponge of suitable size and shape or the like. According to some such embodiments, the mask helps to mitigate the appearance of various skin conditions, including acne, spots, redness and inflammation and/or to decrease the build-up of sebum and toxins on the skin and within the pores of the skin. According to some embodiments, an improvement in the appearance of the skin may be seen after a single application.

According to some embodiments, the skin-care product is contacted with water by immersing the skin-care product in a receptacle (such as a glass or a bowl) containing a premeasured amount of water. According to some embodiments, the skin-care product is contacted with water by first placing the skin-care product in a receptacle and then adding a premeasured amount of water. According to some embodiments. the skin-care product is contacted with water by first placing the skin-care product in a receptacle and then adding an amount of water sufficient to provide a paste of a desired consistency. According to some embodiments, the skin-care product is provided in a kit further comprising a premeasured amount of water and/or a receptacle in which the skin-care product may be contacted with the water. According to some embodiments, once the paste has exited from the bag, additional water may be added to provide a more fluid consistency, if desired.

According to some embodiments, the skin-care product is contacted with water in a bath, whereupon the formed paste, upon exiting the bag, is diluted to form a viscous liquid. According to some such embodiments, the amount of water in the bath is sufficient for the body of the subject to be at least partially immersed in the water in the bath, such that the body of the subject is at least partially coated by the viscous liquid.

According to some embodiments, the paste is applied by pressing on the liquid-permeable container, such as the porous, sealed bag, such that the paste exits the bag, and spreading the paste on a selected portion of the skin, either by hand or by use of any suitable implement.

According to some embodiments, the paste is applied to at least a portion of the skin of the face or body of the subject (such as to an area of the skin of the forehead, nose, cheeks, chin), to substantially the entire face, or to a portion of the skin other than the skin of the face (such as to a portion of the skin of the back, shoulders, neck, hands, elbows, feet, arms, leg, buttocks, or torso).

According to some embodiments, the skin condition is selected from the group consisting of acne, eczema, psoriasis, dermatitis, rosacea, an age-related skin condition (such as wrinkles, age spots), and combinations thereof. According to some embodiments, wherein the skin condition is an age-related condition, the dermatologically effective material is an anti-aging material, such as an anti-aging material selected from the group consisting of a vitamin, an antioxidant, a hydroxy acid, hyaluronic acid, dimethylaminoethanol (DMAE) and combinations thereof.

According to some embodiments, after application of the paste to the portion of skin, the paste is left on the skin for a suitable amount of time (such as about one minute, about two minutes, about five minutes, about ten minutes, about fifteen minutes, about twenty minutes). According to some such embodiments, at the end of the amount of time, the paste is removed by wiping and/or rinsing with water.

According to some embodiments, the paste is for application at any suitable frequency, such as four times per day, three times per day, twice a day, once a day, six times per week, five times per week, four times per week, three times per week, twice per week, once weekly, three times per month, twice per month, once monthly, or as needed. According to a preferred embodiment, the paste is for application twice per week.

### EXAMPLES

### Example 1: Powder Composition

Kaolin clay
Bentonite clay
Rooibos leaves
Rooibos leaf extract
Chamomile flower extract
Salicylic acid
Maltodextrin
Pomegranate extract
Calendula officinalis flower extract
Green tea leaf extract

### Example 2: Face Mask

A powder composition according to Example 1 was prepared and provided to a subject within a porous, sealed bag, in accordance with the principles of the present invention.

About 3 tablespoons of warm water were added to the contents of the bag such that the water was absorbed by the powder composition to provide a paste.

The paste was spread onto the face of the subject and allowed to remain in contact with the skin for about 10 minutes, then washed off.

The subject reported an improvement in the appearance of the skin, including a cleaner and brighter appearance and increased softness.

1. A skin-care product comprising a sealed, liquid-permeable container containing a composition in the form of a powder, said powder comprising a dermatologically effective material, wherein said powder is configured to form a paste upon contact with water, and wherein said container is configured to release at least a portion of said formed paste.

2. The skin-care product according to claim 1, wherein said dermatologically effective material is selected from the group consisting of a dermatologically compatible clay, a dried plant material and/or extract thereof, an antioxidant, a and a keratolytic agent and combinations thereof.

3. The skin-care product according to claim 2, wherein said clay is selected from the group consisting of kaolin clay, bentonite clay, French green clay, French rose clay, Fuller's earth clay, Rhassoul clay and combinations thereof.

4. The skin-care product according to claim 3, wherein said clay comprises bentonite clay and kaolin clay.

5. The skin-care product according to claim 2, wherein said dried plant material and/or extract thereof is selected from the group consisting of rooibos, chamomile, spearmint, nettle, oregano, rosemary, dandelion, sage, arrowroot, ginger, gingko, ginseng, hibiscus, basil, parsley, Calendula officinalis, aloe vera, Echinacea, Comfrey, lavender, violet, thyme, cinnamon, helichrysum, Camellia sinensis and combinations thereof.

6. The skin-care product according to claim 5, wherein said dried plant material and/or extract thereof comprises dried leaves and extract of a rooibos plant, chamomile extract, green tea extract and Calendula officinalis extract.

7. The skin-care product according to claim 2, wherein said antioxidant is selected from the group consisting of vitamin C, niacinamide, resveratrol, vitamin E, retinol, butylated hydroxyanisole (BHA), a carotene, ubiquinol (coenzyme Q), lipoic acid, uric acid, gluthianone, a polyphenol, pomegranate extract and combinations thereof.

8. The skin-care product according to claim 2, wherein said keratolytic agent is selected from the group consisting of salicylic acid, urea, citric acid, polyhydroxy acid, lactic acid, allantoin, glycolic acid, trichloroacetic acid and combinations thereof.

9. The skin-care product of claim 2, comprising pomegranate extract and salicylic acid.

10. The skin-care product according to claim 1, wherein said powder composition comprises kaolin clay, bentonite clay, Aspalathus Linearis (Rooibos) Leaves, Aspalathus Linearis (Rooibos) Leaf Extract, Chamomilla Recutita (Chamomile) Flower Extract, Salicylic Acid, Maltodextrin, Punica Granatum Pericarp (Pomegranate) Extract, Calendula officinalis Flower Extract, Camellia sinensis (Green Tea) Leaf Extract.

11. The skin-care product according to claim 1, wherein said liquid-permeable container comprises a porous, sealed bag.

12. The skin-care product according to claim 11, wherein said bag comprises expandable pores, wherein upon expansion of said pores, said paste exits said bag through said pores.

13. The skin-care product according to claim 12, wherein said pores are expandable upon contact of said bag with water.

14. The skin-care product according to claim 12, wherein said pores are expandable upon exertion of manual pressure upon said bag by a user.

15. The skin-care product according to claim 12, wherein said bag is elastic, thereby expending said pores upon applying traction power thereon.

16. The skin-care product according to claim 12, wherein said paste occupies a greater inner volume of said bag than said powder, and wherein said powder occupies a sufficient inner volume of said bag, such that upon formation of said paste, outward pressure is exerted upon said bag causing expansion of said pores.

17. The skin-care product according to claim 1, for use in treatment of a skin condition.

18. A method of treating a skin condition in a subject in need thereof, comprising providing the skin-care product of claim 1, contacting the skin-care product with water wherein a paste is formed, and applying the paste to at least a portion of the skin of the subject.

19. The skin-care product for use according to claim 17, wherein said skin condition is selected from the group consisting of acne, eczema, psoriasis, dermatitis, rosace, an age-related skin condition, and combinations thereof.

20. The skin-care product for use according to claim 17, wherein said skin condition is a condition of the skin of the face of a subject.

21. The skin-care product for use according to claim 17, wherein said skin condition is a condition of the skin of an area other than the face of a subject.
